(19) United States
(12) Patent Application Publication
(10) Pub. No.: US 2020/0008972 A1
(43) Pub. Date: Jan. 9, 2020

(54) METHOD AND DEVICE FOR PRODUCING AN INTRAUTERINE DEVICE

(71) Applicant: PAT&CO BVBA, Lichtervelde (BE)

(72) Inventor: Dirk Wildemeersch, Ghent (BE)

(21) Appl. No.: 16/490,506

(22) PCT Filed: Feb. 22, 2018
(86) PCT No.: PCT/EP2018/054394

§ 371 Date: Aug. 30, 2019

(30) Foreign Application Priority Data
Mar. 2, 2017 (BE) BE2017/5128

(51) Int. Cl. A61F 6/14; B29C 57/04

(52) U.S. Cl. CPC A61F 6/144; B29C 67/0044; B29C 2793/0027; B29C 57/04

(57) ABSTRACT

In a first aspect, the current invention concerns a method for fabricating an intrauterine device (IUD), said method comprising the step of providing a coaxial rod comprising an outer polymeric layer and a polymeric core, and further comprising the step of cutting from said rod at least one rod segment having a predetermined length, whereby, simultaneously to cutting said rod segment from said rod, the rod end portions thereby formed are compressed into dome-shaped end portions, in such a way that, at said dome-shaped end portions, said polymeric core is at least partly enclosed by said outer polymeric layer. In a second aspect, the current invention concerns a device for cutting a rod segment from a rod.

### TECHNICAL FIELD

The invention pertains to the technical field of fabricating intrauterine devices having a contraceptive action. In particular, the invention relates to a method and a device for the fabrication of such devices.

### BACKGROUND

Contraceptive T- and Q-shaped intrauterine devices (IUDs) have been described earlier, for example in WO 03 068 117. The latter document in particular, discloses a fibrous drug delivery system for the sustained delivery of bioactive agents, more in particular active pharmaceutical ingredients (APIs). These systems are highly interesting for the release of such compounds in the uterus. Similar systems could also be used for subdermal delivery of such compounds.

The shape of IUDs is designed such that they can be inserted and retained in the uterus by themselves. The ease and safety of insertion is thereby of utmost importance. Among other considerations, the IUD design is thus such that the IUD optimally passes through the narrow cervical canal of the uterus. Another important feature is that the IUD can be retained in the uterus in a non-permanent fashion. To this end, the IUD is usually provided with an extraction thread/wire. The device can be extracted from the uterus via said extraction thread/wire, upon exerting a sufficiently large pulling force.

The T-shaped IUD design of WO 03 068 117 is particularly effective. It comprises a transverse arm, constituting the horizontal T-branch, and a stem, constituting the vertical T-branch. The arm is connected to the upper extremity of the stem. Such intrauterine devices can be fabricated as a single piece of molded or extruded material. Alternatively, according to multiple IUD fabrication technologies, the stem and transverse arm are separate rod segments that are attached to each other, thereby forming a device that can be inserted in the uterus, after which it is retained in the uterus by itself. At least one of said rod segments constitutes the so-called drug delivery rod: it comprises the bioactive agent, and thereby acts as a drug delivery system.

Drug delivery systems such as drug delivery rods are increasingly being used for medical applications. They are easy to manufacture via an extrusion process, which is relatively fast. In this process, one or more bioactive agents are first combined with synthetic polymers/plastics, and subsequently extruded to form rods. The extrusion fibers can be processed in a continuous way, which lowers their production cost. Following to extrusion, the extrudate is wound on a roll or cut into rod segments having a predetermined length, before they are assembled in the ultimate IUD devices. For instance, in the case of T-shaped IUD devices, often a crossarm is connected to the upper part of such a drug delivery rod. Said drug delivery rod then makes up the vertical T-branch of the T-shaped device. It comprises a bioactive agent, namely an active pharmaceutical agent (API). The crossarm itself, on the other hand, is not necessarily a drug delivery rod. In a next step, a removal tail is attached to the bottom of the drug delivery rod, allowing withdrawal if the IUD from the uterine cavity if required.

Such drug delivery systems in the form of drug delivery rods are generally considered to be well-controllable and precise. Indeed, particular polymers/plastics can be combined with particular bio-active agents in a particular IUD design, thereby engineering the bioactive agent release rate. First-order and zero-order (constant) rates of release can be obtained, with some systems giving release rates that appear to be a combination of both. Thus, complex pseudo constant release rates may be obtained from some fibrous devices.

Multiple strategies exist for designing drug delivery rods that exhibit a zero-order release rate over a prolonged period of time. In a first approach, the bioactive agent is dissolved or homogenously dispersed within a polymer. Subsequently, a polymer rod segment is formed from said polymer, for example via an extrusion process. Said rod segment is then coated with an outer layer comprising a second polymer.

Alternatively, said outer layer comprises the same type of polymer, now having another bioactive agent concentration. For instance, it comprises no bioactive agent at all. The material, thickness, and structure of the distinct layers drastically affects the release rate of the drug delivery rod. It is thereby possible to obtain a nearly constant release rate, over a prolonged period of time. In this respect, the combination of the inner core and the outer layer, the so-called rate-controlling membrane, is of particular importance. In a second approach, hollow rods are spun and subsequently filled with a material that comprises the bioactive agent. In a third approach, a coaxial rod, having an inner core and an outer layer, is directly extruded in a single co-extrusion process. The polymeric core thereby comprises the bioactive agent, while the outer polymeric layer serves as rate-controlling membrane. This third approach is preferred, because the core and the outer layer are formed simultaneously, during one extrusion process. The (nearly) constant release rate of the bioactive agent is thus achieved without further modification of the rod. It provides for a fast and reliable method for fabricating rod segments comprising a known amount and concentration of bioactive agent.

Of course, for assembling the ultimate IUD devices, rod/fiber segments have to be cut from longer rod/fiber lengths. A disadvantage is that the resulting rod segments have open end portions. The active core material of the resulting IUDs, when inserted into the uterus, is thus directly exposed to the endometrial environment. The release of bioactive agent is therefore heavily localized at said end portions. In some but certainly not all cases, such a highly localized release could be advantageous. However, the overall bioactive agent release rate increases. An important disadvantage is that the lifespan of the drug delivery rod therefore shortens. One solution is to cover the open end portions using a medical adhesive. However, a disadvantage is that this may render insertion into the uterus more difficult. Another disadvantage is that this increases the complexity of the device, which is to be avoided since the IUD has to remain in the uterus for an extended period of time, up to 10 years or more. Yet another disadvantage is that it requires an additional step in the IUD fabrication process and may increase the requirements by regulatory agencies.

Inventors are faced with new challenges to make the fabrication process more efficient and less cumbersome. In particular, the number of fabrication steps should be reduced to a minimum. Additionally, these steps should be versatile, fast, and cost effective. Also, said fabrication methods should allow for cutting drug delivery rod segments that can be assembled to form an IUD. In particular, the amount and the concentration of the bioactive agent in these rod segments should be well-controllable. Additionally, said rod segments should have a drug release rate that can be engineered over an extended period of time, up to 10 years or more.

### SUMMARY

In a first aspect, the present invention discloses a method for fabricating an intrauterine device (IUD) comprised of a stem and a transverse arm, said method comprising the steps of:
providing a coaxial rod, obtainable via co-extrusion of polymers and comprising an outer polymeric layer and a polymeric core, said polymeric core comprising a bioactive composition, and
cutting from said rod at least one rod segment having a predetermined length and suitable for forming said stem and/or said arm;
   in particular, simultaneously to cutting said rod segment from said rod, the rod end portions thereby formed are compressed into dome-shaped end portions, in such a way that, at said dome-shaped end portions, said polymeric core is at least partly enclosed by said outer polymeric layer.

An advantage is that, at said end portions, the core is at least partly enclosed by the outer layer of the coaxial rod. As such, the core is either partly or completely shielded from the environment, at the newly formed end portions. This ensures an more uniform diffusion of the bioactive agent over the entire surface of the rod segment. Additionally, the lifespan of the drug delivery rod increases.

Another advantage is that the present method simultaneously combines two acts in a single method step. On the one hand, said step comprises the act of cutting a rod segment from the rod. On the other hand, said step comprises the act of enclosing the core by the outer layer.

Another advantage is that the newly formed end portions are given the shape of a dome. As such, there are no sharp edges involved. Such sharp edges could irritate or cause damage to tissue, or render insertion difficult when employing the rod as (a part of) an intrauterine or device.

Another advantage is that the end portions of the rod segments are compressed. This advantageously improves their visibility during ultrasound examination.

In a second aspect, the present invention discloses a device for cutting a rod segment from a rod suitable for the production of the stem and transverse arm of an IUD, said device comprising:
a frame, said frame being provided with a rotation shaft, whereby said rotation shaft is hollow, thereby defining a corridor along its length, said corridor being configured for the passage of a rod,
a rotable body being rotably mounted on said frame, about said rotation shaft,
two flyweights, each of both having a light arm, a heavy arm, and a pivot point from which said arms extend, and about which said flyweights are pivotably mounted on said rotable body, and
two roller knife blades, said roller knife blades being rotably mounted on the light arm of said flyweights, such that each of both flyweights is provided with one roller knife blade.

An advantage is that the device enables performing at least one step of the method outlined above, namely the step of cutting a rod segment of a rod.

Another advantage is that, during use of the device for cutting a rod segment from a rod, the roller knife blades exert a constant and well-controllable pressure on the sides of said rod.

### DESCRIPTION OF FIGURES

FIGS. 1a, 1b, and 1c depict three axial cuts of a coaxial rod that is subjected to a possible embodiment of the method according to the present invention.

FIGS. 2a and 2b respectively depict a front view and a side view on a roller knife blade according to an embodiment of the invention.

FIGS. 3a and 3b respectively depict a front view and a side view on a flyweight according to an embodiment of the invention.

FIG. 4 shows an exploded view on the device according to an embodiment of the invention.

FIG. 5 shows a perspective view on the device according to the same embodiment of the invention.

FIGS. 6a-c show longitudinal cuts of three possible embodiments of an extraction wire end portion provided with a fastening head.

FIGS. 7a-c show longitudinal cuts according to an embodiment of the method for fastening an extraction wire into a dome-shaped end portion of a rod segment.

### DETAILED DESCRIPTION

The present invention concerns a method and a device for the fabrication of intrauterine devices (IUDs) having a contraceptive action.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

In a first aspect, the present invention discloses a method for fabricating an intrauterine device (IUD) comprised of a stem and a transverse arm, said method comprising the steps of:
providing a coaxial rod, obtainable via co-extrusion of polymers and comprising an outer polymeric layer and a polymeric core, said polymeric core comprising a bioactive composition, and
cutting from said rod at least one rod segment having a predetermined length and suitable for forming said stem and/or said arm;
   in particular, simultaneously to cutting said rod segment from said rod, the rod end portions thereby formed are compressed into dome-shaped end portions, in such a way that, at said dome-shaped end portions, said polymeric core is at least partly enclosed by said outer polymeric layer. One of said newly formed end portions is comprised by the rod segment cut. The second newly formed end portion is comprised by the remainder of the rod. Both are compressed into dome-shaped end portions, where the polymeric core is at least partly enclosed by the outer polymeric layer, as stated above.

An advantage is that, at said end portions, the core is at least partly enclosed by the outer layer of the coaxial rod. As such, the core is partly or completely shielded from the environment, in particular at the newly formed end portions. This ensures a more uniform release of the bioactive composition over the entire surface of the rod segment. Additionally, the lifespan of the drug delivery rod increases. The instantaneous value of the release rate, as well as its evolution in time, can be engineered by altering the materials and the design of the drug delivery rods. In a non-limiting, further embodiment, the coaxial rod segment comprises a thermoplast, and the abovementioned end portions are slightly heated during the latter method step above. This advantageously improves the act of closing said end portions, and thus of enclosing the core by the outer layer. Said heating could be active heating and/or frictional heating.

Another advantage is that the present method simultaneously combines two acts in a single step. On the one hand, said step comprises the act of cutting a rod segment from the rod. On the other hand, said step comprises the act of enclosing the core by the outer layer, at the newly formed end portions.

Another advantage is that the newly formed end portions are given the shape of a dome. As such, there are no sharp edges involved. Such sharp edges could irritate or cause damage to the uterus, or make insertion difficult, when employing the rod as (a part of) an intrauterine contraceptive device. Alternatively, such edges could irritate or cause damage to skin and/or muscle tissue, when employing the rod as (a part of) a subdermal contraceptive device. Advantageously, the act of reshaping the end portions into the shape of a dome is also performed simultaneously, in the same method step.

Another advantage is that the end portions of the rod segments are compressed. This advantageously improves their visibility during ultrasound examination. In a non-limiting, further or alternative embodiment, metallic particles and/or metal elements or ions are added to the polymer or plastic material matrix, in order to improve visibility during ultrasound examination.

Preferably, the polymeric core comprises a polymer such as a slow-release polymer. Preferably, the outer polymeric layer comprises a polymer such as a slow-release polymer. Suitable non-limiting polymers include polyethylene, polypropylene, polymethylpentene, ethylene/propylene copolymers, ethylene/ethyl acrylate copolymers, ethylene/vinyl acetate (EVA) copolymers, polycarbonate, polytetrafluoroethylene (PTFE), fluoroethylenepropylene (FEP), polyvinylidene fluoride (PVDF), polyvinylacetate, polystyrene, polyamides, polyurethane, polybutadiene, polyisoprene, chlorinated polyethylene, polyvinyl chloride, vinyl chloride copolymers with vinyl acetate, poly(methacrylate), polymethyl (meth)acrylate, poly(vinylidene) chloride, poly(vinylidene) ethylene, poly(vinylidene) propylene, polyethylene terephthalate, ethylene vinylacetate, a polyhydroxy alkoanate poly(lactic acid), poly(glycolic acid), poly(alkyl 2-cyanoacrylates), polyanhydrides, polyorthoesters, ethylene/vinyl alcohol copolymer, ethylene/vinyl acetate/vinyl alcohol terpolymer; ethylene/vinyloxyethanol copolymer, hydrophilic polymers such as the hydrophilic hydrogels of esters of acrylic and methacrylic acids, modified collagen, cross-linked polyvinyl alcohol, cross-linked, partially hydrolyzed polyvinyl acetate, silicone elastomers, especially the medical grade polydimethyl siloxanes, polyvinylmethylsiloxanes, other organopolysiloxanes, polysiloxane, neoprene rubber, butyl rubber, epichlorohydrin rubbers, hydroxyl-terminated organopolysiloxanes of the room temperature vulcanizing type which harden to elastomers at room temperature following the addition of cross-linking agents in the presence of curing catalysts, two-component dimethylpolysiloxane compositions which are platinum catalysed at room temperature or under elevated temperatures and capable of addition cross-linking as well as mixtures thereof. Especially suitable polymers are an elastomer composition comprising poly(dimethylsiloxane) (PDMS), an elastomer composition comprising a siloxane-based elastomer comprising 3,3,3-trifluoropropyl groups attached to the Si-atoms of the siloxane units, an elastomer composition comprising poly(alkylene oxide) groups, said poly(alkylene oxide) groups being present as alkoxy-terminated grafts or blocks linked to the polysiloxane units by silicon-carbon bonds, or as a mixture of these forms and a combination of at least two thereof. More preferably, the polymer is selected from the group consisting of an ethylene/vinyl acetate (EVA) copolymer, polyurethane (PU), and silicone. More preferably, the polymeric core comprises a polymer selected from the group consisting of an ethylene/vinyl acetate (EVA) copolymer, polyurethane (PU), and silicone. More preferably, the outer membrane comprises a polymer selected from the group consisting of an EVA copolymer, PU, and silicone.

Of course, the coaxial rod may also be called a coaxial fiber; the terms “fiber” and “rod” may be interchanged. In any case, the outer diameter of the rod may range from 0.25 to 10 mm. It is preferred that the outer diameter is bigger than 0.50 mm, yet smaller than 5 mm. It is more preferred that the outer diameter at least 1 mm, yet no more than 2.9 mm. Such rods thus have radii ranging from 0.5 mm to 1.45 mm. The rod is wound on a roll or cut into shorter rod segments. Preferably, the length of the rod segments is between 1 cm and 10 cm. More preferably, the length of the rod segments is between 2.5 cm and 4.1 cm.

The term “coaxial rod”, as employed throughout this document, should be interpreted in a very broad sense. For instance, the coaxial rod may have a core and two or more coaxial layers, said layers cladding the inner core. Alternatively, it is not readily possible to identify the distinct layers of the coaxial rod, because there is no clear separation between said layers. For instance, the coaxial rod is made of one material that serves as a matrix hosting the bioactive composition. The concentration of said bioactive composition is thereby maximal near the rod axis, and takes lower values for points that lie further away from said axis. Other in-diffused or extruded concentration profiles can be imagined by the person skilled in the art. Alternatively, any of the layers can comprise any bioactive composition listed hereunder, or any suitable bioactive composition known in the art.

The bioactive composition may comprise, consist essentially of, or consist of at least one active pharmaceutical ingredient (API). The composition may comprise, consist essentially of, or consist of at least one non-active pharmaceutical ingredient. The composition may comprise a combination of at least one active and at least one non-active pharmaceutical ingredient.

The at least one active pharmaceutical ingredient may be selected from the group consisting of contraceptives, hormones, steroids, drugs for hormone replacement therapy, selective androgen receptor modulators (SARM), drugs for the treatment of premenstrual syndrome, drugs for the treatment of endometriosis, drugs for the treatment of uterine fibroids including uterine leiomyomata and leiomyosarcoma, drugs for cervical ripening and/or induction of labour, selective estrogen receptor modulators (SERMs), selective progestin receptor modulators (SPRM), antimalarial substances, osteoporosis drugs, antiprogestins, aromatase inhibitors, bone active substances, antiurinary incontinence substances, serotonin reuptake inhibitors (SSRIs), drugs for genitourinary disorders, antiemetic drugs, 5HT3 antagonists, anti-angiogenesis factors, growth factors, enzymes, anesthetics, analgesics, anticoagulants and thrombolytic substances, anti-inflammatory substances, antimicrobials, anti-protozoal substances, antiviral substances, neuroleptic and antipsychotic drugs, opiate antagonists and agonists, anti-fibroid substances, antihypertensives, angiotensin inhibitors, anti-protozoal substances, anti-addiction drugs, anti-angiogenesis factors, anti-bacterial substances, anticancer chemotherapeutic substances, antifungals, antioxidants, diuretics, drugs for the central nervous system, fibrinolytic substances, free radical scavengers, gene therapy substances, growth factors, neuro-trophic factors, peptides, photodynamic therapy substances, proteins, symphatomimetic substances, thrombin inhibitors, thrombolytic substances, and a combination of at least two thereof. The at least one active pharmaceutical ingredient may be a medicament such as a medicament for use in the treatment of menorrhagia, pain and/or other conditions such as conditions confined to the intrauterine cavity.

The composition may comprise a second active pharmaceutical ingredient, for example a substance capable of preventing or suppressing abnormal and/or irregular endometrial bleeding. Hence, the at least one active pharmaceutical ingredient may be selected from the group of prostaglandin synthesis inhibitors, NSAIDs, inhibitors of leukotriene, oxytocin antagonists, pancreatic trypsin inhibitors, COX-inhibitors, antifibrinolytic drugs, estrogens, antiestrogens, aromatase inhibitors, cytokine inhibitors, glucocorticoids, progestogens with pronounced glucocorticoid acticity, danazol, gestrinone, and angiogenesis inhibitors.

Suitable, non-limiting contraceptives include levonorgestrel (LNG), desogestrel, or progesterone. Suitable non-limiting hormones include estrogens, progestogens, glucocorticoids and mineralocorticoids; analogs, agonists and antagonists thereof. For example, suitable estrogens include tamoxifen, oestrogen, oestradiol, ethinyl oestradiol, estron, quinestranol, estriol, estetrol and mestranol. For example, suitable progestogens include progesterone, dienogest, medroxyprogesterone acetate, norgestrel, levonorgestrel, norethindrone, norethindrone acetate, desogestrel, norgestimate, and ethynodiol diacetate, norelgestromin, 30 norethisteron, dydrogesteron, drospirenon, 3-beta-hydroxydesogestrel, etonogestrel, 17-deacetylnorgestimat, 19-norprogesteron, acetoxypregnenolon, allylestrenol, amgeston, chlormadinon, cyproteron, demegeston, dihydrogesteron, dimethisteron, ethisteron, ethynodioldiacetat, flurogestonacetat, gastrinon, gestoden, gestrinon, hydroxymethylprogesteron, lynestrenol, mecirogeston, megestrol, melengestrol, nomegestrol, norethynodrel, norgestrienon, normethisteron, quingestanol, (17alpha)-17-hydroxy-II-methylen-19-norpregna-4, 15-dien-20-yn-3-on, tibolon, trimegeston, algeston acetophenid, nestoron, promegeston, 17-hydroxyprogesteronester, 19-nor-17-hydroxyprogesteron, 17alpha-ethinyl-testosteron, 17alpha-ethinyl-19-nor-testosteron, d-17-beta-acetoxy-13beta-ethyl-17alpha-ethinyl-gon-4-en-3-onoxim, tanaproget. Preferably, said progestogen is progesterone or levonorgestrel (LNG).

Preferably, the composition comprises at least one active pharmaceutical ingredient selected form levonorgestrel (LNG), desogestrel, or progesterone. More preferably, the composition comprises the active pharmaceutical ingredient levonorgestrel. In certain embodiments, the stem element may comprise a polymer selected from the group consisting of ethylene/vinyl acetate (EVA) copolymer, polyurethane (PU), and silicone and may comprise a composition comprising at least one active pharmaceutical ingredient selected form levonorgestrel (LNG), desogestrel, or progesterone, preferably LNG.

According to a preferred, further embodiment of the method, said rod segment is cut from said rod by two opposing knife blades which encircle said rod in a transverse plane, while radially approaching the rod axis. The term “transverse plane” thereby comprises any plane that is orthogonal to the rod axis. The terms “radial” and “radially” thereby relate to a “radial direction” with respect to the rod axis, being any direction orthogonal to said axis. Preferably, the knife blades continue to approach the rod axis, up to the point where the two blades touch. The rod segment is then effectively cut from the remainder of the rod. Preferably, the knife blades exert a controllable pressure on the rod.

An advantage of having such roller blades encircling and radially approaching the rod axis, is that a 360° incision is made gradually and uniformly on the outer side of the rod.

According to a preferred, further embodiment of the method, said knife blades are roller knife blades. An advantage is that a more precise and neat cut can be made, because such roller knife blades exert only a radial force on the rod axis.

Advantageously, it is not necessary to actuate the roller knife blades themselves: their rolling movement is an immediate result of friction with respect to the rod.

Alternative methods, employing linear knife blades, might slightly crush the rod. It is preferred that the (radially opposing) knives exert an equal but opposing force on the rod, such that the rod does not bend while it is cut. This allows for a high precision cutting process. However, alternatively, linear knife blades might be employed.

According to a preferred, further embodiment of the method, said roller knife blades have a cutting edge portion, the 2D profile of which is symmetric and comprises a cutting tip that is flanked by a hollow, quarter circle leg at either side.

An advantage of using such roller knife blades is that, simultaneously to cutting a rod segment from the remainder of the rod, the thereby formed end portions can be shaped into the shape of a dome. At the same time, the core is at least partly enclosed by the outer layer.

Of course, the term “quarter circle” should be interpreted in a broad sense, comprising but not limited to circle and ellipse quadrants. The term “dome” should equally be interpreted in a broad sense, comprising but not limited to all kinds of spherical and ellipsoid caps.

According to a preferred, further embodiment of the method, the radius of curvature of said legs is between 0.25 and 5 mm. Preferably, said radius of curvature ranges from 0.5 mm to 1.45 mm. Even more preferably, said radius of curvature is not smaller than the radius of curvature of the rod to be cut.

According to a preferred, further embodiment of the method, said method additionally comprises the step of connecting a transverse arm to a rod segment, said rod segment forming said IUD stem. Preferably, the thus obtained connected structure (being the IUD) has a form that advantageously can be retained in the uterus by itself. Preferably, said transverse arm comprises polyethylene (PE). More preferably, said transverse arm is made of injection molded PE.

Alternatively, the IUD has a T-shaped form, in which a first rod segment constitutes the stem of the T, while a second rod segment constitutes a transverse arm. Further rod segments, connected to said stem, may constitute additional transverse arms.

According to a non-limiting embodiment, the rod segment constituting the stem of the T is provided with a transverse channel at its upper part, and the arms are made up of a rod segment having a section lower than the one of the stem but higher than that the one of the channel, inserted forcibly into said channel to form two equal arms on both sides of the IUD, the rod segment forming the arms presents a curved form and the wedging force exercised by the stem on the latter is such that it is usually retained in the channel in the position where it has been introduced, that is to say a position where the arms are oriented downwards of the stem, while it is permitted to pivot in the channel until the arms are oriented upwards of the stem under the effect of the constraints exercised on the arm during the extraction process of the device from the womb. The channel intended to receive the arm on the upper part of the body may be formed by drilling and the rod segment constituting the arm is introduced by force into said channel. Optionally, due to the pressing force involved, there is no heavily localized release of the bioactive composition. Alternatively, only the transverse arm (the core remains of which remains enclosed by the outer layer, in the current embodiment) has the coaxial structure and comprises the bioactive composition.

According to a non-limiting, alternative embodiment, the stem of the T presents a cavity on its upper part in which the rod segment constituting the arm is housed, and in which the rod segment is attached to the body by gluing. The cavity intended to receive the arms on the upper part of the body may be formed by grooving, the rod segment constituting the arms is introduced there and is held there by gluing. Optionally, due to the glue, there is no heavily localized release of bioactive composition. Alternatively, only the transverse arm (the core remains of which remains enclosed by the outer layer, in the current embodiment) has the coaxial structure and comprises the bioactive composition.

According to a non-limiting, alternative embodiment, one or more coupling elements are included, configured for the attachment between rod segments. Said attachment may or may not be dismountable. For instance, a coupling element is included for attaching the transverse arm of the T to the stem of the T. Said coupling element may comprise a passage configured to fittingly/slidably receive both the stem rod segment and the transverse arm rod segment. However, other coupling elements may be envisaged by the man skilled in the art. In the current embodiment, the stem and/or the transverse arm may have a coaxial structure, the core of which remains enclosed by the outer layer.

According to a non-limitative, alternative embodiment, the rod segment is employed as such, for instance as a subdermal contraceptive device.

According to a preferred, further embodiment of the method, said method additionally comprises the step of connecting an extraction wire to the rod segment forming said IUD stem. An advantage is that the IUD can be extracted from the uterus via said extraction wire, upon exerting a sufficiently large, pulling force. Alternatively, the extraction wire is attached to the coupling element.

In a second aspect, the present invention discloses a device for cutting a rod segment from a rod suitable for the production of the stem and transverse arm of an IUD, said device comprising:
a frame, said frame being provided with a rotation shaft, whereby said rotation shaft is hollow, thereby defining a corridor along its length, said corridor being configured for the passage of a rod,
a rotable body being rotably mounted on said frame, about said rotation shaft,
two flyweights, each of both having a light arm, a heavy arm, and a pivot point from which said arms extend, and about which said flyweights are pivotably mounted on said rotable body, and
two roller knife blades, said roller knife blades being rotably mounted on the light arm of said flyweights, such that each of both flyweights is provided with one roller knife blade.

In this respect, the term “heavy arm” and the term “light arm” should further be elucidated. In fact, the flyweight above is one integral body that comprises a “light arm” and a “heavy arm”. The “heavy arm” is not necessarily heavier than the “light arm”. However, its moment of inertia (or angular mass) with respect to the pivot point is greater—hence the nomenclature employed. Upon rotation of the rotable body, the flyweights will pivot with respect to the rotable body, about their pivot point, provided that the rotation speed is sufficiently high. The centrifugal force will then be sufficiently large. The separation between their heavy arm and the axis of rotation thereby increases, while their light arm conversely approaches the axis of rotation. Since the roller knife blades are rotably mounted on the light arms of the flyweights, said roller knife blades likewise approach the axis of rotation.

An advantage is thus that the device can be used for cutting a rod segment from a coaxial rod similar to the ones described above. Such a rod is then provided along the aforementioned axis of rotation, through the corridor of the hollow rotation shaft. The rod is thus approached by radially opposing circle knife blades that encircle it in a transverse plane. The device thus enables performing at least one step of the method outlined above.

Another advantage is that, during use of the device, the roller knife blades exert a well-controllable pressure on the sides of the rod. This pressure value is directly related to the rotation speed and to the specific design of the flyweights. Latter factors can be adequately engineered by the man skilled in the art, taking into account the materials comprised by the rod, among other things.

Of course, the design of the flyweights can be a lot more complex, in which case the definition of the “light arm” and the “heavy arm” should be generalized. The “heavy arm” then relates to those part (or parts) of the flyweight that move away from the axis of rotation, when the rotable body acquires a sufficiently high rotation speed. Conversely, “light arm” then relates to those part (or parts) of the flyweights that approach the axis of rotation. According to a non-limiting embodiment, the device comprises a retention spring that is such that, when the rotation speed is sufficiently low (or zero), the heavy arms are retracted and thus maximally approach the axis of rotation. At the same time, the separation between the roller knife blades and the axis of rotation is then maximal. However, in other embodiments no such spring is included.

Preferably, the rotation axes of the rotable body, the pivot axes of the flyweights, and the rotation axes of the roller knife blades are all parallel to one another. Preferably, the frame is also sufficiently heavy, such that the stability of the device is increased. Preferably, the pivot points of the flyweights are positioned non-centrally with respect to the rotable body—the pivot points thus not coincide with the axis of rotation of the rotable body. Advantageously, the flyweights will then pivot about their pivot point as described above, on the condition that the rotation speed is sufficiently high.

Any rotable and/or pivot connection mentioned above, for connecting two members, may include but is not limited to: a screw-and-nut or bolt-and-nut connection through sets of mutually aligned bore holes in either of both members, a pin comprised by one member through a hole in the other member, a screw or bolt through sets of mutually aligned bore holes, at least one of them being a threaded bore, and any other connection means known in the state of the art. Optionally, said connections are secured using securing means such as but not limited to retaining rings, retaining pins, and thread lockers. Any of these connections may also comprise more advanced bearings such as ball bearings or roller bearings but not limited thereto.

According to a preferred, further embodiment of the device, said roller knife blades have a cutting edge portion, the 2D profile of which is symmetric and comprises a cutting tip that is flanked by a hollow, quarter circle leg at either side. According to a preferred, further embodiment of the device, the radius of curvature of said legs is between 0.5 and 5 mm. Preferably said roller knife blades are the same as the ones described above. They therefore bring about the same advantages, in particular when the device is used for cutting a rod segment from a rod as described by the method outlined above. Also, the more detailed specifications of the roller knife blades are the same as described above. The person skilled in the art is capable of choosing a suitable radius of the blades and radius of curvature of the quarter circle legs, thereby taking into account the rod radius and the material(s) comprised by the rod.

According to a preferred, further embodiment of the device, the diameter of said corridor is between 0.5 to 10 mm. Preferably the diameter is greater than 5 mm. More preferably said diameter is at least as big as the diameter of the thickest rod to be cut. Preferably, said corridor is provided centrally. Preferably, said corridor is configured for the passage of rods as introduced above.

An advantage is that a rod can be provided through this corridor. The axis of the rod then coincides with the axis of rotation of the rotable body. Said rod is thus optimally positioned for being cut by the roller knife blades above, whereby the rod is approached by two radially opposing roller knife blades that encircle it in a transverse plane. Said blades exert a controllable pressure on the rod and make a 360° incision of a gradually increasing depth. The number of encirclements is at least such that the rod segment is effectively cut from the remainder of the rod, whereby the newly formed end portions are compressed into dome shaped end portions.

Preferably, the hollow rotation shaft is connected to the frame at one end portion. The opposite end portion is a free end portion. There is enough space at the latter (free) end portion for providing the assembly of the rotable body, flyweights, and roller knife blades. However, other configurations are not excluded.

According to a preferred, further embodiment of the device, said flyweights and said roller knife blades are positioned on said rotable body in a center-symmetrical configuration. Preferably, the flyweights are either substantially identical or symmetrical. Preferably, the roller knife blades are substantially identical. Such a center-symmetrical configuration then advantageously improves the stability of the device by reducing vibrations during the rotation of the rotable body.

According to a preferred, further embodiment of the device, said frame is additionally provided with at least one gutter; said gutter is positioned in line with said corridor. For instance but not limited thereto, said gutter is U-shaped or V-shaped. The expression “in line with” should be understood such that, for the average rod diameter, the axis of the rod that is provided in the gutter coincides more or less with the axis of rotation of the rotable body. An advantage is that such gutters are suitable for supporting a rod, a rod segment, and/or a remainder of a rod, whereby the rod is originally inserted in the gutter, through said corridor, where it can be radially encircled by two approaching roller knife blades. The rod, rod segment and/or remainder of the rod is thereby positioned centrally with respect to the axis of rotation. Preferably, two gutters are provided, one at either side of the rotable body. More preferably, said two gutters are positioned in line with said rotation shaft.

According to a preferred, further embodiment of the device, said device additionally comprises a ruler and an adjustable marker that is configured for retaining an end portion of a rod. Such a marker advantageously allows for setting a certain length for rod segments to be cut. Preferably, the length of the rod segments is between 1 cm and 10 cm. More preferably, the length of the rod segments is between 2.5 cm and 4.1 cm.

According to a preferred, further embodiment of the device, said frame is additionally provided with an electrical engine for driving said rotable body and with an electrical switch for operating said engine. An advantage is that this is a very convenient way for rotating the rotable body. Preferably, it is possible to set a precise value of the rotation speed, depending on the specifics of the rod, the flyweights, and the roller knife blades. Alternatively, the standard engine rotation speed is adequate for cutting the most frequently employed type of rod. Note that it is not necessary to provide for additional means for actuating the roller knife blades: these simply roll along the sides of the rod.

According to a preferred, further embodiment of the device, said frame, said rotable body, said flyweights, and/or said roller knife blades are made from stainless steel. An advantage is that stainless steel is not prone to oxidation.

According to a preferred, further embodiment of the device, the motor and/or the assembly comprising the rotable body, the flyweights, and the roller knife blades are shielded by some kind of cap, for instance by a stainless steel cap. An advantage is that this renders the device safer.

According to a preferred, further embodiment of the invention, the device above is employed for cutting a rod segment from a rod according to the method above.

In a third aspect, the present invention relates to a method for fabricating an intrauterine device (IUD) comprised of a stem and a transverse arm, and provided with an extraction wire, said method comprising the step of providing at least one rod segment having an outer polymeric layer and a polymeric core, said polymeric core comprising a bioactive composition, and further providing an extraction wire having an end portion; said method further comprises the steps of:
creating a channel through said rod segment, said channel having a channel entry and a channel exit,
subjecting the end portion of said extraction wire to a heated air stream having a maximal temperature superior to 100° C.,
inserting said extraction wire through said channel, via said channel entry.

An advantage is that this method allows for fastening an extraction wire to an IUD in a simple yet effective fashion. Another advantage is that this fastening method does not result in wire loops or wire knots that protrude with respect to the IUD, such that it is more comfortable, both to wear and during insertion. In addition, a knot could result in accumulation of mucus and tissue that can infect. Furthermore, when inserting the IUD using a hysteroscope, a hysteroscope having a smaller inserter tube can be employed, further simplifying the insertion procedure.

The channel can be created by material removal, for instance by providing a bore hole, or by material deformation, for instance by forcing a needle through the rod segment material.

Preferably, said rod segment is as described above. Preferably, the extraction wire has a diameter between 0.1 mm and 1.0 mm, more preferably between 0.2 and 0.5 mm, more preferably between 0.3 mm and 0.4 mm. Most preferably, the diameter of the extraction wire is between 0,310 mm and 0,390 mm.

The inventors surprisingly found that, when subjecting the end portion of the extraction wire to the heated air stream, the partly molten material agglomerates such that on one hand the extraction wire shortens, while on the other hand a widened end portion, the so-called fastening head, is formed by the agglomerated material. The extraction wire is then cut to length, which can be done either prior to or following to the formation of said fastening head. The opposite end portion of the extraction wire is inserted into the abovementioned channel entry, after which said extraction wire is pulled through said channel op to the point where its fastening head reaches the channel entry, thereby prohibiting further pulling through the extraction wire. An advantage is that this fastening method is simple yet effective.

The fastening technology set out above is in contrast with popular practices in the state of the art. According to most of latter practices, the extraction wire forms a loop through a channel in the rod segment. For instance, said channel extends radially through the rod segment. The loop is usually closed by a knot. Such a wire loop and/or knot protrude, and might therefore irritate the uterine tissue and make insertion difficult. Also, this is inconvenient and uncomfortable during insertion of the IUD through the cervical canal. An advantage of the present method is that it does not involve such protruding loops or knots, thereby being more comfortable for the patient and easier to insert.

Preferably, said fastening head has a diameter superior to the diameter of the extraction wire, whereby the “diameter of the fastening head” is considered equal to the maximum (transverse) diameter value across said fastening head. More preferably, said diameter measures between 0.5 mm and 3 mm, more preferably between 1.0 mm and 2.0 mm, and most preferably said diameter is between 1.2 mm and 1.5 mm. Preferably, said fastening head is mushroom-shaped.

The longer the material is heated, the more material contracts towards the remainder of the thread because of cohesion, and the bigger the fastening head. However, for heating times that are too long and/or for heating temperatures that are too high, fastening heads of an irregular and less controllable shape are obtained.

Preferably, said channel is formed by forcing a needle to the rod segment material. Preferably, the diameter of said needle is a few tens of millimeters bigger than the diameter of the extraction wire, such that the channel (at least initially) allows for an easy passage of the extraction wire. In the most preferred situation, the diameter of the extraction wire is between 0,310 mm and 0,390 mm, while the diameter of the curved needle is between 0.4 mm and 0.8 mm, even more preferably equal to about 0.8 mm. Preferably, said needle is a curved needle.

Preferably, the extraction wire has a length between 10 mm and 30 mm, more preferably between 15 mm and 25 mm, and most preferably between 18.2 mm and 22.0 mm. The latter length allows for easy insertion and extraction of the IUD, and for a maximum comfort during and after insertion.

In a further preferred embodiment of the method, said extraction wire comprises polypropylene (PP). An advantage is that the PP material readily allows for creating mushroom-shaped fastening heads of regular shape and size, by subjecting said PP wire to a heated air stream. Preferably, said extraction wire is a monofilament PP wire. Preferably but not limited thereto, said PP extraction wire comprises a coloring agent such as a Cu-phtalocyanine dye.

Preferably, the maximal temperature of the air stream is superior to 100° C.; more preferably, it is superior to 150° C., more preferably 200° C., more preferably 250° C., more preferably 300° C., more preferably 350° C. This maximal temperature should be lower than 500° C. Preferably, the extraction wire end portion is heated during a predetermined amount of time, typically several seconds to several tens of seconds. Preferably, said amount of time is between 1 second and 40 seconds, more preferably between 2 seconds and 20 seconds, and even more preferably between 3 seconds and 16 seconds. Most preferably, said amount of time is about equal to 9.5 seconds.

In a further preferred embodiment of the method, said heated air stream is directed axially. In this paragraph, the term axially refers to the direction in line with the end portion of the extraction wire. An advantage of employing an axially directed, heated air stream is that the resulting fastening head has a cylinder-symmetrical, regular shape.

In a further preferred embodiment of the method, said method additionally comprises the step of widening said channel entry. Preferably, the channel entry is widened prior to channel formation by means of drilling (material removal), yet this order can be reversed. Also, other techniques can be employed, for instance heat-assisted deformation methods. An advantage of widening the channel entry is that the fastening head is conveniently nested into said widened channel entry, such that it no longer protrudes. This provides an easier insertion of the IUD. Additionally, it is more comfortable for the subject. Preferably, said widened channel entry has an inner diameter about equal to the outer diameter of said fastening head. More preferably, said inner diameter is equal to said outer diameter.

In a further preferred embodiment of the method, said channel exit is positioned axially. The term “axial” hereby relates to directions in line with the axis of the rod segment (that will ultimately be) constituting the IUD stem. An advantage is that the extraction wire axially extends from the rod segment, in line with said rod segment. Therefore, upon extraction of the IUD via its extraction wire, said IUD will not be tilted.

In a further preferred embodiment of the method, said channel entry is positioned radially. The term “radial” hereby relates to directions orthogonal to the axis of the rod segment (that will ultimately be) constituting the IUD stem. Thereby, the channel can follow a curved or a straight line between its entry and its exit. An advantage is that the channel is as short as possible, such that it minimally damages the inner core of the rod segment.

The method of the third aspect, may thereto be described according to the following embodiments:
1. A method for fabricating an intrauterine device (IUD) comprised of a stem and a transverse arm, and provided with an extraction wire 38, said method comprising the step of providing at least one rod segment 8 having an outer polymeric layer 5 and a polymeric core 4, said polymeric core 4 comprising a bioactive composition, and further providing an extraction wire 38 having an end portion; said method further comprises the steps of:
   creating a channel 43 through said rod segment, said channel 43 having a channel entry 44 and a channel exit 45,
   subjecting said end portion of said extraction wire 38 to a heated air stream having a maximal temperature superior to 100° C., such that a fastening head 39 is formed at said end portion, and
   inserting said extraction wire 38 through said channel 43, via said channel entry 44.
2. The method according to previous embodiment 1, characterized in that said extraction wire 38 comprises polypropylene (PP).
3. The method according to any of embodiments 1 and 2, characterized in that said heated air stream is directed axially.
4. The method according to any of embodiments 1 to 3, characterized in that said method additionally comprises the step of widening said channel entry 44.
5. The method according to any of embodiments 1 to 4, characterized in that said channel exit 45 is positioned axially.
6. The method according to any of embodiments 1 to 5, characterized in that said channel entry 44 is positioned radially.

The aspects of the current invention will further be elucidated by means of examples and figures, without being limited to these examples or figures.

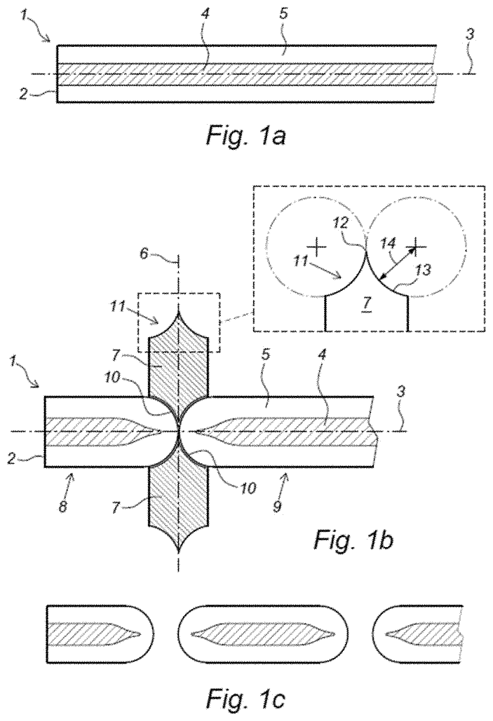

FIGS. 1a, 1b, and 1c depict three axial cuts of a coaxial rod 1 that is subjected to a possible embodiment of the method according to the present invention.

FIG. 1a shows an axial cut of the rod 1, said rod having a flat end portion 2. The plane of the axial cut comprises the rod axis 3, which defines the “axial direction”. “Radial directions” are defined as all directions that are orthogonal to said axial direction. The coaxial rod 1 comprises a core (comprising a core 4), and an outer layer (comprising an outer layer 5).

FIG. 1b shows an axial cut of the same rod 1, whereby said rod 1 is encircled, in a transverse plane 6, by two radially opposing roller knife blades 7. While encircling the rod 1, said roller knife blades 7 radially approach the rod axis 1, up to the point where a rod segment 8 is effectively cut from the remainder of the rod 9. Two new end portions 10 are thereby introduced: one belonging to the rod segment 8 and another belonging to the remainder of the rod 9. Both of said end portions 10 are dome-shaped, due to the particular form of the cutting edge portion 11 of the roller knife blades 7. Additionally, at said dome-shaped end portions 10, the core 4 is enclosed by the outer layer 5. The inset of the figure shows an enlarged 2D profile (or cut) of such a cutting edge portion 11. The cutting tip 12 is flanked by two hollow, quarter circle legs 13, one at either side of the cutting tip 12. The radius of curvature 14 of said legs 13 can be chosen within the interval given above, yet preferably its value is about equal to the radius of the coaxial rod 1.

FIG. 1c shows an axial cut of the same rod 1, whereby multiple rod segments 8 have been cut from said rod 1. The first rod segment 8 has one flat end portion 2 and one dome-shaped end portion 10. From the second rod segment 8 onwards, each rod segment 8 has dome-shaped end portions 10 at either side. As such, their core 4 is effectively enclosed by their outer layer 5.

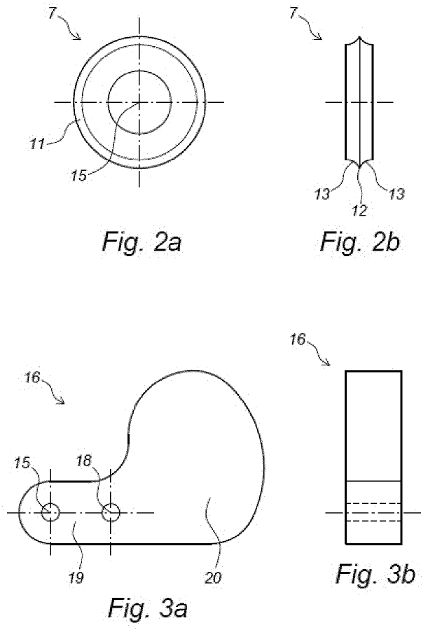

FIGS. 2a and 2b respectively depict a front view and a side view on a roller knife blade 7 according to an embodiment of the invention. The roller knife blade 7 has the form of a disk, and is provided with a rotation bore 15. Via said rotation bore 15, the roller knife blade 7 can be rotably mounted on another member, such as the flyweight 16 hereunder. The cutting edge portion 11 of the blade 7 has a particular form: in profile, it comprises a cutting tip 12 that is flanked by two hollow, quarter circle legs 13, as can be seen on FIG. 2b. The roller knife blade 7 is preferably made from stainless steel, yet it can be made from any other material, such as but not limited to a metal other than stainless steel, or a ceramic.

FIGS. 3a and 3b respectively depict a front view and a side view on a flyweight 16 according to an embodiment of the invention. The flyweight 16 is preferably made from stainless steel, yet it can be made from any other material, such as a metal other than stainless steel, or a ceramic. The flyweight 16 comprises a pivot point, about which it can be mounted pivotably on another member, such as the rotable body 17 hereunder. In the present embodiment, the flyweight 16 is provided with a pivot bore 18 at its pivot point. The flyweight 16 also comprises a light arm 19 and a heavy arm 20, both extending from said pivot point. The light arm 19 of the flyweight 16 is provided with a (threaded) rotation bore 15, similar to the rotation bore 15 of a roller knife blade 7, yet with internal thread. The rotation bore 15 of the flyweight 16 serves for the rotable connection with a roller knife blade 7, via the rotation bore 15 of said roller knife blade 7.

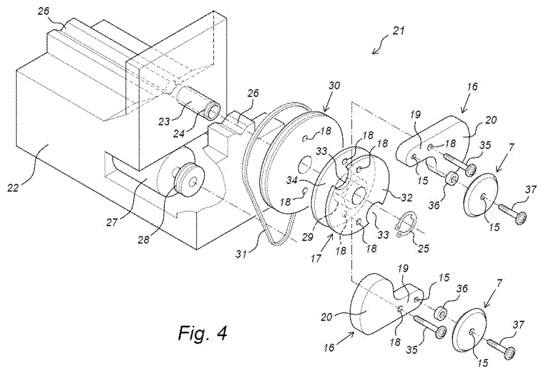

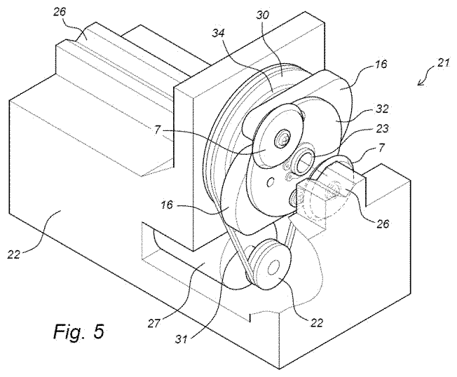

FIG. 4 shows an exploded view on the device 21 according to an embodiment of the invention. The device 21 comprises a frame 22 that is provided with a hollow rotation shaft 23. Said rotation shaft 23 has a proximal end portion, via which it is connected to the frame 22, and a distal end portion, opposite to its proximal end portion. At its free, distal end portion, the rotation shaft 23 is externally provided with a machined groove 24, for co-action with a retaining ring 25 such as a C-Clip or an E-Clip. The rotation shaft 23 is hollow; it is provided with a corridor along its length, whereby said corridor is configured for the passage of a (coaxial) rod 1. The frame 22 is further provided with two V-shaped gutters 26, in line with the corridor of the rotation shaft 23, at either side of said rotation shaft 23. Each of both gutters 26 is thereby configured for receiving and supporting an end portion of the rod 1. The frame 22 is also provided with an electrical engine 27, driving a driver pulley 28, and with means for operating said engine 27, such as an electrical switch. The device 22 further comprises a rotable body 17. Said rotable body 17 comprises a tube segment 29 via which it is rotably mounted on the frame 22, about the rotation shaft 23. The rotable body 17 is connected to a driven pulley 30, for instance using screws. Alternatively, the rotable body 27 and the driven pulley 30 form one integral part. The driven pulley 30 is driven by the driver pulley 28 by means of a belt 31, for instance a rubber or nylon belt 31, or a belt made from any other suitable material. The retaining ring 25 permits rotation of the rotable body 17 about the rotation shaft 23, yet it prevents any axial movement. The device 21 additionally comprises two flyweights 16. For the sake of embedding said flyweights 16 sideways, both ends of tube segment 29 of the rotable body 17 are provided with wings 32/34. The distal wing 32 has the form of a disk; its edge is provided with two radially opposing indentations 33. The proximal wing 34 simply has the form of a disk, without indentations. The flyweights 16 have a pivot point, about which they are pivotably mounted on the rotable body 17. To this end, they are provided with a pivot bore 18 at their pivot point. The driven pulley 31 and the wings 32/34 of the rotable body 17 have similar sets of pivot bores 18. Said distinct pivot bores 18 can be brought in line with each other. Two pivot screws 35 (e.g. made from stainless steel) then provide for a pivotable connection between the flyweights 16 and the rotable body 17. Their second function is to fix the driven pulley 30 to the rotable body 17. At least one pair of pivot bores 18, for instance the ones comprised by the driven pulley 30, are threaded, such that said pivot screws 35 can be fixed. Preferably, some kind of thread locker, such as a thread-locking fluid, is used for locking said pivot screws 35 into said threaded pivot bores 18. As mentioned above, the flyweights 16 have a light arm 19 and a heavy arm 20; both extend from the pivot point. The device 22 additionally comprises two roller knife blades 7 that are rotably connected to the light arm 19 of the flyweights 16. This way, each flyweight 16 is provided with one roller knife blade 7. In order to provide for such a rotable connection, the light arm 19 of each flyweight 16 is provided with a threaded rotation bore 15. The roller knife blades 7 are rotably fixed into said threaded rotation bore 15 by means of a rotation screw 37. Additionally, some kind of tubular distance holder 36 is included, such that the roller knife blades 7 protrude the distal wing 32 of the rotable body 17. Preferably, said rotation screw 37 is made of stainless steel.

Also, it might be preferred to use some kind of thread locker, such as a thread-locking fluid, for locking said rotation screws 37 into said rotation bores 15. Bearings, such as roller bearings or ball bearings, may be included in rotably fixing the roller knife blades 7 to the light arms 19 of the flyweights 16.

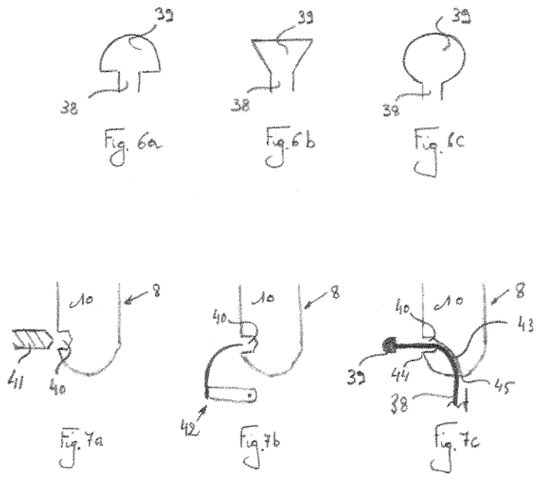

FIG. 5 shows a perspective view on the device 21 according to the same embodiment of the invention. Suppose that a rod 1 is provided through the rotation shaft 23 corridor. Either end of said rod 1 is supported by a gutter 26. Alternatively, a long rod 1 is wound on a roll, and said roll is set up in line with the rotation shaft 23 corridor. When actuating the electrical engine 27, the driver pulley 28 starts to rotate. Said rotation is transferred to the driven pulley 30 via the rubber belt 31. The rotable body 17 is directly connected to the driven pulley 30 and thus rotates as well. Provided that the rotation speed is sufficiently high, the flyweights 16 start to pivot with respect to the rotable body 17. This is because of the centrifugal force, thereby noting that the angular mass of the heavy arm 20 exceeds the angular mass of the light arm 19 with respect to the pivot point. The separation increases between the axis of rotation and the heavy arms 20 of the flyweights 16. At the same time, the light arms 19 of the flyweights 16 pivotably approach the axis of rotation. The roller knife blades 7 likewise approach the axis of rotation, and the distance holders 36 niche themselves into the indentations 33 in the distal wing 32 of the rotable body 17. A rod segment 8 is thereby cut from the remainder of the rod 9. The engine 27 is then switched off. In some embodiments, the roller knife blades 7 move back to their original position. However, this is not necessarily so.

According to a non-limiting example, the rods have a diameter between 1.0 mm and 2.9 mm, and standard length of about 33 cm. They additionally have flat, open end portions at either side. Said rods are cut into rod segments having a predetermined length, between 2.5 cm and 4.1 cm. In order to do so, an adjustable marker is correctly installed with respect to a ruler thereto provided, to a chosen value between 2.5 cm and 4.1 cm. In a next step, a rod is placed in the long gutter, in line with the rotation shaft. Said rod is then passed through the corridor in the rotation shaft. Its protruding end portion is at least partly supported by the short gutter, and retained against the adjustable marker. In this respect, the adjustable marker determines the length of the rod segment that is cut. At this point, the electrical engine is switched on, and a first rod segment is cut from the rod. The legs in the cutting profile of the roller knife blades have a radius of curvature of about 1.5 mm, thus always greater than the radius of the rods to be cut. Said first rod segment is kept apart: it has one flat, open end portion and one dome-shaped, closed end portion. Said dome-shaped end portion is dome-shaped in the sense that it has the form of a spherical cap, but not necessarily the form of a hemisphere. The electrical engine is switched off, causing the roller knife blades to move back to their original position, away from the axis of rotation. The remainder of the rod is then passed along, until its newly formed end portion is retained against the adjustable marker. The electrical engine is switched on again, and a second rod segment is cut from the rod. Said second rod segment has two dome-shaped, closed end portions, in addition to a correct length. It can be used for fabricating an intrauterine device. Alternatively, it could be used directly as a subdermal contraceptive device. These steps are repeated until the last rod segment is cut. The last rod segment is kept apart: it has one flat, open end portion and additionally it does not necessarily have the correct length.

According to a non-limiting example, the IUD is provided with a monofilament polypropylene (PP) extraction wire, having a diameter ranging from 0,310 mm to 0,390 mm.

For the purpose of connecting the extraction wire to the IUD, said extraction wire is provided with a widened fastening head. In order to do so, a massive, brass block is provided that has a tunnel going through it, said tunnel having a tunnel entry and a tunnel exit. Alternatively, this block can be made of stainless steel, or any other material having both a relatively high thermal conductivity and a relatively high thermal capacity. The extraction wire snugly fits into the latter tunnel, and it is inserted into said tunnel such that an end portion of about 5 mm in length protrudes at the tunnel exit. A heat gun is directed towards said protruding end portion, in line with said channel and said protruding end portion. The heat gun (preferably timer-controlled) is activated during a predetermined time of approximately 9.5 seconds, thereby producing a heated air stream having a nominal air temperature of 400° C. and a nominal air volume of 300 I/min. A Bosch PHG 600-3 (1800 W) heat gun is employed. The protruding end portion of the extraction wire is subjected to this heated air stream, such that it partly melts. As a consequence of the material properties of PP, the heated PP material agglomerates because of cohesion forces. The extraction wire thus becomes shorter and the agglomerated material gathers at its end portion, thereby forming a widened fastening head, having the shape of a mushroom. Alternatively, the fastening head may be cone-shaped, spherical, or it may have any other shape. In any case, the fastening head can be used for fastening the extraction wire to an IUD, as will be described below. In particular, it withstands knot pulling forces up to at least 20 N, preferably even up to 50 N. During the formation of the fastening head, the brass block has the function of absorbing all excess heat, and of shielding the remainder of the extraction wire from the heated air stream, such that overheating of said PP material is avoided. Overheated PP material has the tendency of adhering to other objects it is in contact with, which would result in soiling of the equipment. In case of a continuous fabrication process, where tens or hundreds of fastening heads are subsequently formed, the one after the other, this is particularly important. Optionally, resting periods should be introduced at regular time intervals, thereby giving said brass block the opportunity to cool down. It is further preferred that the brass block is provided with cooling fins and/or an active cooling system, such as a Peltier cooling system and/or active ventilation or a cooling circuit comprising a refrigerant. Further in case of a continuous fabrication process, a bobbin holding the PP thread is incorporated, for the sake of providing a continuous PP thread supply. Fastening heads are formed on the threads and said threads are cut to length to form extraction wires—which may occur in any order. Extraction wires typically have a total length between 18.2 cm and 22.0 cm. Preferably, the equipment employed in fabricating the extraction wires additionally comprises a cutting blade, a ruler and an adjustable marker. This allows for efficiently cutting the extraction wires to the desired length.

A rod segment as described above is now provided. Either said rod segment constitutes the stem of a T-shaped IUD, or it is suitable for doing so. In a next step, the extraction wire as described above is fastened to an end portion of said rod segment, being the lower end portion of said stem in the former case. Thereto, first the rod segment is radially provided with a shallow bore hole, at a height of about 2.2 mm from the rod segment end portion. At a later stage, said shallow bore hole will function as a widened channel entry. Subsequently, a curved needle is forced radially into said rod, thereby entering the rod segment at the position of said bore hole, curving downwards, and exiting the rod segment axially at the rod segment end portion. A quarter circle channel having a widened channel entry that is positioned radially, and a channel exit that is positioned axially, is thus formed. The diameter said curved needle should be such that the channel created is wide enough for the passage of the 0,310 mm to 0,390 mm diameter PP extraction wire. Therefore, the diameter of the curved needle is between 0.4 and 0.8. Subsequently to forming the channel, the needle is withdrawn and the extraction wire is inserted via its end portion opposite to its fastening head. The extraction wire is then pulled through said channel, such that its fastening head is nested in the widened channel entry. The fastening head thereby partly shields the inner core of the rod segment. Since the rod segment material is elastic, the aforementioned channel will partly close again, further fastening the extraction wire to the rod segment. Optionally, the rod segment is subsequently rolled over a flat surface while exerting some pressure, thereby flattening all surface irregularities, and thereby further pressing the fastening head into the widened channel entry.

FIGS. 6a-c show longitudinal cuts of three possible embodiments of an extraction wire 38 end portion provided with a fastening head 39. FIG. 6a shows a longitudinal cut comprising a mushroom-shaped fastening head 39. FIG. 6b shows a longitudinal cut comprising a cone-shaped fastening head 39. FIG. 6c shows a longitudinal cut comprising a spherical fastening head 39.

FIGS. 7a-c show longitudinal cuts according to an embodiment of the method for fastening an extraction wire 38 into a dome-shaped end portion 10 of a rod segment 8. First, a radially-oriented, shallow bore hole 40 is made into the surface of said rod segment 8. To this end, a drill 41 is employed. In a second step, a pivotable, curved needle 42 is forced through said rod segment 8, via said shallow bore hole 40. By pivotably forcing said needle 42 into the rod segment 8 material, a curved channel 43 is formed. Said channel 43 has a widened channel entry 44 that is positioned radially and a channel exit 45 that is positioned axially with respect to said rod segment 8. In a last step, the extraction wire 38 is inserted into said curved channel 43, such that its fastening head 39 will be nested into the widened channel entry 44. For sake of simplicity, the multilayered core and outer layer structure of the rod segment is not shown in the figures.

It is supposed that the present invention is not restricted to any form of realization described previously and that some modifications can be added to the presented example of fabrication without reappraisal of the appended claims.

### ELEMENTS ON FIGURES

1. Rod
2. Flat end portion
3. Rod axis
4. Core
5. Outer layer
6. Transverse plane
7. Knife blade
8. Rod segment
9. Remainder of the rod
10. Dome-shaped end portion
11. Cutting edge portion
12. Cutting tip
13. Quarter circle leg
14. Radius of curvature
15. Rotation bore
16. Flyweight
17. Rotable body
18. Pivot bore
19. Light arm
20. Heavy arm
21. Device
22. Frame
23. Rotation shaft
24. Machined groove
25. Retaining ring
26. Gutter
27. Electrical engine
28. Driver pulley
29. Tube segment
30. Driven pulley
31. Belt
32. Distal wing
33. Indentations
34. Proximal wing
35. Pivot screw
36. Distance holder
37. Rotation screws
38. Extraction wire
39. fastening head
40. shallow bore hole
41. drill
42. curved needle
43. curved channel
44. channel entry
45. channel exit

1. A method for fabricating an intrauterine device (IUD) comprising a stem and a transverse arm, said method comprising:
providing a coaxial rod, obtainable via co-extrusion of polymers and comprising an outer polymeric layer and a polymeric core, said polymeric core comprising a bioactive composition, and
cutting from said rod at least one rod segment having a predetermined length and suitable for forming said stem and/or said arm,
characterized in that, simultaneously to cutting said rod segment from said rod, the rod end portions thereby formed are compressed into dome-shaped end portions, in such a way that, at said dome-shaped end portions, said polymeric core is at least partly enclosed by said outer polymeric layer.

2. The method according to previous claim 1, characterized in that said rod segment is cut from said rod by two opposing knife blades which encircle said rod in a transverse plane, while radially approaching the rod axis.

3. The method according to previous claim 2, characterized in that said knife blades are roller knife blades.

4. The method according to previous claim 3, characterized in that said roller knife blades have a cutting edge portion, the 2D profile of which is symmetric and comprises a cutting tip that is flanked by a hollow, quarter circle leg at either side; the radius of curvature of said legs is between 0.25 and 5 mm.

5. The method according to claim 1, characterized in that said method additionally comprises the step of connecting a transverse arm to a rod segment, said rod segment forming said IUD stem.

6. The method according to claim 1, characterized in that said method additionally comprises the step of connecting an extraction wire to the rod segment 8 forming said IUD stem.

7. A device for cutting a rod segment from a rod suitable for the production of the stem and transverse arm of an IUD, said device comprising:
a frame, said frame being provided with a rotation shaft, whereby said rotation shaft is hollow, thereby defining a corridor along its length, said corridor being configured for the passage of a rod,
a rotable body being rotably mounted on said frame, about said rotation shaft,
two flyweights, each of both having a light arm, a heavy arm, and a pivot point from which said arms extend, and about which said flyweights are pivotably mounted on said rotable body, and
two roller knife blades, said roller knife blades being rotably mounted on the light arm of said flyweights, such that each of both flyweights is provided with one roller knife blade.

8. The device according to claim 7, characterized in that said roller knife blades have a cutting edge portion, the 2D profile of which is symmetric and comprises a cutting tip that is flanked by a hollow, quarter circle leg at either side; the radius of curvature of said legs is between 0.25 and 5 mm.

9. The device according to claim 7, characterized in that the diameter of said corridor is between 0.5 to 10 mm.

10. The device according to claim 7, characterized in that said flyweights and said roller knife blades are positioned on said rotable body in a center-symmetrical configuration.

11. The device according to claim 7, characterized in that said frame is additionally provided with at least one gutter;
said gutter is positioned in line with said corridor.

12. The device according to claim 7, characterized in that said device additionally comprises a ruler and an adjustable marker.

13. The device according to claim 7, characterized in that said frame is additionally provided with an electrical engine for driving said rotable body.

14. The device according to claim 7, characterized in that said frame, said rotable body, said flyweights, and/or said roller knife blades are made from stainless steel.
